(19) United States
(12) Patent Application Publication
(10) Pub. No.: US 2017/0181941 A1
(43) Pub. Date: Jun. 29, 2017

(54) SUNSCREEN FORMULATION WITH TUNABLE ABSORPTION AND EMISSION

(71) Applicant: International Business Machines Corporation, Armonk, NY (US)

(72) Inventors: Oki Gunawan, Westwood, NJ (US); Huan Hu, Yorktown Heights, NY (US); Ning Li, White Plains, NY (US); Devendra K. Sadana, Pleasantville, NY (US)

(21) Appl. No.: 14/979,631

(22) Filed: Dec. 28, 2015

(51) Int. Cl. A61K 8/19; A61K 8/29; A61K 8/26; A61Q 17/04; A61K 8/27

(52) U.S. Cl. CPC A61K 8/19; A61Q 17/04; A61K 8/27; A61K 8/26; A61K 8/29; A61K 2800/413; Y10S 977/774; B82Y 5/00

(57) ABSTRACT

Tunable sunscreen compositions include quantum confined nanoparticles selected from the group of zinc oxide nanoparticles, titanium oxide nanoparticles, and gallium nitride-based nanoparticles. The zinc oxide nanoparticles have particle sizes that provide either quantum confined absorption at wavelengths of 290 nm to about 400 nm or emission at about 275 nm to about 290 nm. Still further, the zinc oxide with the quantum confined absorption provides transmission of radiation at wavelengths of about 275 nm to about 290 nm.

### BACKGROUND

The present invention relates to a sunscreen formulation with tunable absorption and/or emission, and more particularly, to a sunscreen formulation including nanoparticles quantum confined absorption and/or emission that can be effectively tuned for ultraviolet radiation absorption and/or emission.

Exposure of human skin to sunlight is an ongoing concern and presents significant health risks including sunburn, as well as the development of melanomas and other forms of skin cancer. Moreover, extended exposure to sunlight can also accelerate the natural aging process in the skin. In addition, ever increasing concerns have been raised about the need to provide both ultraviolet A (UVA) and ultraviolet B (UVB) protection as each has known or suspected health risks. For example, UVA (i.e., wavelength 320 nm-400 nm) radiation is known to penetrate deeply into the dermis and has been widely implicated in damage leading to premature skin aging. Prolonged exposure to UVA radiation is also believed to be carcinogenic and causes alterations to the immune system, which may lead to susceptibility to cancer in the form of basal and squamous cell carcinomas, and possibly even melanomas. On the other hand, UVB radiation (i.e., wavelength 280 nm-320 nm), which consists of around 5-10% of the total ultraviolet radiation that reaches the Earth's surface, is also carcinogenic, and can lead to the development of non-melanoma (basal and squamous cell) carcinomas and malignant melanoma-type skin cancers. UVB radiation is the primary cause of the often painful erythemal reaction associated with sunburn and photo-aging, and recently has been shown to be implicated in a suppression of the immune system which can lead to increased outgrowth of skin tumors. UVB radiation does not penetrate the skin beyond the epidermis.

### SUMMARY

Disclosed herein are sunscreens formulations including nanoparticles exhibiting quantum confined absorption and/or emission of wavelengths in the ultraviolet radiation region. In one embodiment, the sunscreen formulation includes nanoparticles exhibiting quantum confined absorption selected from the group of zinc oxide, gallium-based nitride, titanium dioxide, and combinations thereof, wherein the quantum confined absorption has an average particle size effective to absorb radiation at wavelengths from about 290 nm to 400 nm and provide transmission of wavelengths of about 275 nm to about 290 nm.

In another embodiment, nanoparticles exhibiting quantum confined emission selected from the group of zinc oxide, gallium-based nitride, titanium dioxide, and combinations thereof, wherein the quantum confined absorption has an average particle size effective to emit radiation at wavelengths of wavelengths of about 275 nm to about 290 nm.

In yet another embodiment, nanoparticles with a quantum confined absorption or emission at wavelengths from about 280 nm to 400 nm selected from the group consisting of zinc oxide, indium gallium nitride, aluminum gallium nitride, titanium dioxide, and combinations thereof, wherein the nanoparticles have an average diameter of 1 nm to 2 μm, wherein the zinc oxide nanoparticles are greater than 0 to less than 20 weight percent; wherein the indium gallium nitride nanoparticles and aluminum gallium nitride nanoparticles are greater than 0 to less than 10 weight percent; and the titanium dioxide nanoparticles are greater than 0 to less than 10 weight percent, wherein the weight percent is based on total weight of the formulation.

### DETAILED DESCRIPTION

The present invention is generally directed to sunscreen formulations including particles exhibiting quantum confined absorption and/or emission. By way of example, the particles may include zinc oxide nanoparticles, gallium nitride-based material nanoparticles, titanium dioxide nanoparticles, and various combinations thereof, wherein the wavelength emission and absorption of the sunscreen formulation can be tuned by selection of particle size so as to enable beneficial wavelengths to be transmitted or emitted to the skin and harmful wavelengths absorbed by the sunscreen formulations.

Quantum dots (QDs) are luminescent nanoparticles of semiconductor material, with average diameters typically in the range of 1 nanometer (nm) to 2 microns (m). Their photo-absorption and -luminescence (emission) can generally be tuned by manipulating the particle size. The unique optical and electronic properties of QDs originate from quantum confinement effects; as the QD diameter decreases the electron and hole wave functions become quantum confined, giving rise to discrete energy levels similar to those observed in atoms or molecules, resulting in an increase in the semiconductor band gap with decreasing QD diameter. Accordingly, as the particle size decreases, the nanoparticles can become optically transparent and yet offer the same level of UV absorption as larger particles. For example, zinc oxide can exhibit quantum confinement at relatively small particle sizes and provide the same ultraviolet protection as larger sized zinc oxide particles (e.g., greater than about 1 μm) but without leaving a white residue on the skin associated with the larger sized particles. The high absorption coefficient enables strong absorption from a tiny amount of material.

Using zinc oxide quantum confined nanoparticles as an example, methods to synthesize zinc oxide nanoparticles have been the subject of several reviews, with methods including plasma pyrolysis, combustion synthesis, precipitation reactions, citrate gel synthesis, micro-emulsion synthesis and sol-gel processing. An exemplary process for forming the zinc oxide nanoparticles with quantum confined emission or absorption generally includes reacting a zinc nanoparticle precursors in the presence of a population of molecular cluster compounds as described in US Pat. Pub. No. 2015/0076494. The molecular cluster compound may or may not contain the same metal as will be present in the metal oxide nanoparticle. Likewise, the molecular cluster compound may or may not contain oxygen. The molecular cluster compounds acts a seeds or templates upon which nanoparticle growth is initiated. As the molecular cluster compounds are all identical, the identical nucleation sites result in highly monodisperse populations of metal oxide nanoparticles.

An average diameter of the nanoparticles with quantum confined emission or absorption may generally be in a range of about 1.0 to about 2 μm. The nanoparticles may have a Stokes shift value of about 20 nm or greater between absorption energy and emission energy.

By “absorption” it is meant that the sunscreen compositions absorb the majority of ultraviolet light at wavelengths across the range of both UVA and UVB in most embodiments, at least greater than about 60 percent absorption at all wavelengths across the range of both UVA and UVB radiation in other embodiments, and at least greater than about 80 percent absorption at all wavelengths across the range of both UVA and UVB in still other embodiments. In some embodiments of the present invention, by selection of the particular ingredients and amounts within the sunscreen compositions, the sunscreen compositions may absorb nearly all of the ultraviolet light (i.e., greater than about 95 percent) at wavelengths across the range of both UVA and UVB.

In still other embodiments, the sunscreen formulation is formulated with the above described nanoparticles wherein the quantum confined particles are selected to provide transmission or emission at about 275 nm to about 290 nm of UVB radiation.

In one embodiment, the sunscreen formulation is formulated with zinc oxide, titanium dioxide, and/or gallium nitride-based nanoparticles having particle sizes configured to provide quantum confined emission at wavelengths of about 275 nm to about 290 nm. By way of example, or the quantum confined emission of zinc oxide, the average particle size can be from 1 nm to 2 μm, from 3 nm to 200 nm in other embodiments, from 1 nm to 10 nm in still other embodiments, and 1 to 5 nm in yet other embodiments. Doping the nanoparticles can provide additional wavelength tuning.

In one embodiment, the sunscreen formulation is formulated with nanoparticles configured to provide quantum confined absorption. In this embodiment, the particle size is selected to provide absorption of radiation at wavelengths of greater than about 290 to about 400 nm and transmission of radiation at about 275 nm to about 290 nm. By way of example, for the quantum confined absorption of zinc oxide, the average particle size can be from 1 nm to 2 μm, from 3 nm to 200 nm in other embodiments, and from 3 nm to 10 nm in still other embodiments. Again, doping can be used to provide additional wavelength tuning.

Titanium dioxide and the gallium-based nitrides exhibiting quantum confined absorption will have generally similar average particles sizes as that described above. Likewise, doping of the gallium based nitrides and titanium dioxide can provide additional tuning for the sunscreen formulation.

In the above embodiments, exposure of human skin to UVB radiation at wavelengths of about 275 to 290 nm results in the beneficial production of vitamin D, which is naturally produced when the skin is exposed to UVB radiation at wavelengths of about 275 nm to about 290 nm that is strong enough to penetrate the epidermal layers of the skin. As vitamin D is naturally present in very few foods, sufficient UVB exposure and/or supplementation is important to maintaining proper Vitamin D levels. The present invention provides a sunscreen formulation that permits production of vitamin D by ultraviolet exposure via transmission or quantum confined emission yet prevents harmful ultraviolet radiation form penetrating the epidermis.

Vitamin D is a fat-soluble vitamin that is essential to a number of biological processes, including promoting calcium absorption in the gut, maintaining adequate serum calcium and phosphate concentrations (to enable normal bone mineralization and to prevent hypocalcemic tetany), and is needed for bone growth and bone remodeling by osteoblasts and osteoclasts (van den Berg, H. (1997) Eur. J. Clin. Nutr. 51:S76-9; Institute of Medicine, Food and Nutrition Board. (1997) Dietary Reference Intakes: Calcium, Phosphorus, Magnesium, Vitamin D, and Fluoride. Washington, D.C.: National Academy Press; Cranney C. et al. (2007) Evidence Report/Technology Assessment No. 158 prepared by the University of Ottawa Evidence-based Practice Center under Contract No. 290-02.0021. AHRQ Publication No. 07-E013. Rockville, Md.: Agency for Healthcare Research and Quality). Insufficient vitamin D may result in thin, brittle, and/or misshapen bones, and is a cause of rickets in children and osteomalacia in adults (DeLuca H. F. (2004) Am. J. Clin. Nutr. 80:1689S-96S; Goldring et al. (1995) Endocrinology. 3rd ed. Philadelphia: W B Saunders, 1204-27; Favus M. J. and Christakos S. (1996) Primer on the Metabolic Bone Diseases and Disorders of Mineral Metabolism. 3rd ed. Philadelphia, Pa.: Lippincott-Raven). Together with calcium, vitamin D also helps protect older adults from osteoporosis.

Vitamin D also has other roles in human health, including modulation of neuromuscular and immune function and reduction of inflammation. Many genes encoding proteins that regulate cell proliferation, differentiation, and apoptosis are modulated in part by vitamin D (van den Berg (1999); Cranney et al. (2007); Holick, M. F. (2003) Cancer Res. 164:3-28; Hayes et al. (2003) Cell. Mol. Biol. 49:277-300). Vitamin D deficiency has been linked to breast, prostate and colon cancer, as well as autoimmune diseases such as multiple sclerosis, fibromyalgia, rheumatoid arthritis, Grave's disease, lupus, and others. High serum vitamin D3 levels have also been linked to a reduced risk for skin cancers (See Roehr. B. Dermatology Times. July 2009, p. 24)

As noted above, the sunscreen formulations including the following active ingredients: zinc oxide particles, a gallium nitride-based particles, and titanium dioxide particles. The zinc oxide particles with the quantum confined absorption or emission is generally greater than zero to 20 weight percent of the sunscreen formulation.

The titanium oxide particles are greater than zero to 10 weight percent of the sunscreen formulation.

Suitable gallium nitride based materials include, without limitation, indium gallium nitride, aluminum gallium nitride and combinations thereof. The gallium nitride based particles are greater than zero to 10 weight percent of the sunscreen formulation. With regard to indium gallium nitride, the indium concentration ranges from greater than zero to about 15 percent by weight of the sunscreen formulation.

A total thickness of the applied sunscreen formulation ranges from a few tens of microns to hundreds of microns. The sunscreen formulation effectively blocks ultraviolet radiation at wavelengths less than 390 nm with the exception of wavelengths at about 275 nm to about 290 nm, which corresponds to the wavelengths associated with the production of vitamin D. The transmission is enabled by tuning the size of the particles to tune the energy levels for selective light transmission and fluorescence. The blocking is done by absorption of the active ingredients described above.

Sunscreen compositions according to the present invention can optionally include one or more inactive adjuvants, such as pH adjusters, emollients, humectants, conditioning agents, moisturizers, chelating agents, propellants, rheology modifiers and emulsifiers such as gelling agents, colorants, fragrances, odor masking agents, UV stabilizer, preservatives, and any combination of any of the foregoing. Examples of pH adjusters include, but are not limited to, aminomethyl propanol, aminomethylpropane diol, triethanolamine, triethylamine, citric acid, sodium hydroxide, acetic acid, potassium hydroxide, lactic acid, and any combination thereof.

Suitable conditioning agents include, but are not limited to, cyclomethicone; petrolatum; dimethicone; dimethiconol; silicone, such as cyclopentasiloxane and diisostearoyl trimethylolpropane siloxy silicate; sodium hyaluronate; isopropyl palmitate; soybean oil; linoleic acid; PPG-12/saturated methylene diphenyldiisocyanate copolymer; urea; amodimethicone; trideceth-12; cekimonium chloride; diphenyl dimethicone; propylene glycol; glycerin; hydroxyalkyl urea; tocopherol; quaternary amines; and any combination thereof.

Suitable preservatives include, but are not limited to, chlorophenesin, sorbic acid, disodium ethylenedinitrilotetraacetate, phenoxyethanol, methylparaben, ethylparaben, propylparaben, phytic acid, imidazolidinyl urea, sodium dehydroacetate, benzyl alcohol, methylehloroisothiazolinone, methylisothiazolinone, and any combination thereof. The sunscreen composition generally contains from about 0.001% to about 20% by weight of preservatives, based on 100% weight of total sunscreen composition. In another aspect, the composition contains from about 0.1% to about 10% by weight of preservatives, based on 100% weight of total sunscreen composition.

In one aspect sunscreen compositions according to the present invention include a water phase. These sunscreen compositions can also optionally include any cosmetically acceptable solvent. Non-limiting examples of such solvents can include hydrocarbons, alcohols, esters and blends thereof.

The necessary amount of the adjuvants, additives and/or additional active ingredients can, based on the desired end product, easily be chosen by a person skilled in the art.

In some modes of practicing the invention, the sunscreen formulation provides a buffered environment, preferably an environment buffered to a pH of between about 4.0 to about 9.0, such as a pH of about 4.5, 5.0, 5.5, 6.0, 6.5, 7.0, 7.5, 8.0 or 8.5.

As noted above, the tunable sunscreen compositions can be tuned to provide protection from UVB radiation, UVA radiation, or both, with selective transmission or quantum confined emission at about 175 nm to about 190 nm for vitamin D production. Likewise, depending on the particle sizes for the other components, the sunscreen formulation can be transparent or opaque. One type of sunscreen composition employs physical screening agents, e.g., titanium oxide or zinc oxide. These particles have sometimes been applied in the form of thick, opaque creams that diffuse and scatter UV radiation. Where the particle sizes have been large (generally above about 0.1 micron), the resulting opacity (“whitening”) can diminish aesthetic appeal.

Still other aspects, features, and technical effects will be readily apparent to those skilled in this art from the following detailed description, wherein preferred embodiments are shown and described, simply by way of illustration of the best mode contemplated. The disclosure is capable of other and different embodiments, and its several details are capable of modifications in various obvious respects. Accordingly, the drawings and description are to be regarded as illustrative in nature, and not as restrictive.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one more other features, integers, steps, operations, element components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The diagrams depicted herein are just one example. There may be many variations to this diagram or the steps (or operations) described therein without departing from the spirit of the invention. For instance, the steps may be performed in a differing order or steps may be added, deleted or modified. All of these variations are considered a part of the claimed invention.

While the preferred embodiment to the invention had been described, it will be understood that those skilled in the art, both now and in the future, may make various improvements and enhancements which fall within the scope of the claims which follow. These claims should be construed to maintain the proper protection for the invention first described.

1. A sunscreen formulation comprising:
nanoparticles exhibiting quantum confined absorption including zinc oxide, gallium-based nitride, and titanium oxide, wherein the quantum confined absorption has an average particle size effective to absorb radiation at wavelengths from about 290 nm to 400 nm and provide transmission of wavelengths of about 275 nm to about 290 nm.

2. The sunscreen formulation of claim 1, wherein the zinc oxide nanoparticles are greater than zero to about 20 weight percent, the titanium oxide nanoparticles are greater than zero to about 10 weight percent, and the gallium nitride-based nanoparticles are greater than zero to about 10 weight percent, wherein the weight percent is based on total weight of the formulation.

3. The sunscreen formulation of claim 1, wherein the gallium nitride-based nanoparticles are selected from the group consisting of indium gallium nitride, aluminum gallium nitride, and mixtures thereof.

4. The sunscreen formulation of claim 3, wherein the indium is at a concentration of greater than zero to 15% of the sunscreen formulation.

5. The sunscreen formulation of claim 1, wherein the formulation upon application is transparent.

6. The sunscreen formulation of claim 1, wherein the formulation upon application is at a thickness of about 10 μm to about one thousand μm.

7. The sunscreen formulation of claim 1, wherein the zinc oxide nanoparticles with the quantum confined absorption is at the average particle size of 3 to 10 nm.

8. The sunscreen formulation of claim 1, wherein the sunscreen formulation provides a buffered environment between about 4.0 to about 9.0.

9. The sunscreen formulation of claim 1, wherein the particles exhibiting quantum confined absorption are doped to further tune radiation absorption.

10. The sunscreen formulation of claim 1, further comprising an inactive adjuvant selected from the group consisting of pH adjusters, emollients, humectants, conditioning agents, moisturizers, chelating agents, propellants, rheology modifiers and emulsifiers such as gelling agents, colorants, fragrances, odor masking agents, UV stabilizer, preservatives, and combinations thereof.

11. A sunscreen formulation comprising:
nanoparticles exhibiting quantum confined emission including zinc oxide, gallium-based nitride, and titanium oxide, wherein the quantum confined emission has an average particle size effective to emit radiation at wavelengths of wavelengths of about 275 nm to about 290 nm.

12. The sunscreen formulation of claim 11, wherein the zinc oxide nanoparticles are greater than zero to about 20 weight percent, the titanium oxide nanoparticles are greater than zero to about 10 weight percent, and the gallium nitride-based nanoparticles are greater than zero to about 10 weight percent, wherein the weight percent is based on total weight of the formulation.

13. The sunscreen formulation of claim 11, wherein the gallium nitride-based nanoparticles are selected from the group consisting of indium gallium nitride, aluminum gallium nitride, and mixtures thereof.

14. The sunscreen formulation of claim 13, wherein the indium is at a concentration of greater than zero to 15% of the sunscreen formulation.

15. The sunscreen formulation of claim 11, wherein the formulation upon application is transparent.

16. The sunscreen formulation of claim 11, wherein the formulation upon application is at a thickness of about 10 micron to about one thousand micron.

17. The sunscreen formulation of claim 11, wherein the zinc oxide nanoparticles with the quantum confined emission is at the average particle size of 1 nm to 2 μm.

18. The sunscreen formulation of claim 11, wherein the sunscreen formulation provides a buffered environment between about 4.0 to about 9.0.

18. (canceled)

19. The sunscreen formulation of claim 11, further comprising an inactive adjuvant selected from the group consisting of pH adjusters, emollients, humectants, conditioning agents, moisturizers, chelating agents, propellants, rheology modifiers and emulsifiers such as gelling agents, colorants, fragrances, odor masking agents, UV stabilizer, preservatives, and combinations thereof.

20. A sunscreen formulation comprising:
nanoparticles with a quantum confined absorption or emission at wavelengths from about 280 nm to 400 nm including zinc oxide, indium gallium nitride, aluminum gallium nitride, and titanium oxide, wherein the nanoparticles have an average diameter of 1 nm to 2 μm, wherein the zinc oxide nanoparticles are greater than 0 to less than 20 weight percent; wherein the indium gallium nitride nanoparticles and the aluminum gallium nitride nanoparticles are greater than 0 to less than 10 weight percent; and the titanium oxide nanoparticles are greater than 0 to less than 10 weight percent, wherein the weight percent is based on total weight of the formulation.

21. The sunscreen formulation of claim 11, wherein the zinc oxide nanoparticles with the quantum confined absorption is at the average particle size of 3 nm to 10 nm.
